(19) United States
(12) Patent Application Publication
(10) Pub. No.: US 2012/0329945 A1
(43) Pub. Date: Dec. 27, 2012

(54) BENZOXAZINE-CONTAINING COMPOSITIONS

(75) Inventors: Atsushi Mori, Osaka (JP); Atsushi Sudo, Tokoyo (JP); Takeshi Endo, Yokohoma (JP); Ryoichi Kudoh, Shiga (JP); Andreas Taden, Duesseldorf (DE); Rainer Schoenfeld, Duesseldorf (DE); Thomas Huver, Duesseldorf (DE); Iris Christa Huver, (US); Stefan Kreiling, Eppelheim (DE)

(73) Assignee: Henkel AG & Co. KGaa, Duesseldorf (DE)

(21) Appl. No.: 13/458,731

(22) Filed: Apr. 27, 2012

Related U.S. Application Data
(63) Continuation of application No. PCT/EP2009/064150, filed on Oct. 27, 2009.

(51) Int. Cl. C07C 271/58; C08L 79/04; C07C 271/44; C08G 73/06

(52) U.S. Cl. 524/611; 528/210; 524/876; 560/32; 560/26

(57) ABSTRACT

The present invention relates to a curable composition, comprising specific meta-substituted aromatic compounds and at least one benzoxazine compound. In particular, the invention relates to the use of said meta-substituted aromatic compounds as curatives/catalysts for benzoxazine-containing compositions.

### FIELD OF THE INVENTION

The present invention relates to a curable composition, comprising specific meta-substituted aromatic compounds and at least one benzoxazine compound. In particular, the invention relates to the use of said meta-substituted aromatic compounds as curatives/catalysts for benzoxazine-containing compositions.

### DESCRIPTION OF THE PRIOR ART

Normally, benzoxazines are cured at relatively high temperatures. In order to reduce the polymerization temperature of benzoxazines various curatives, like phenols (JP2000-178332A), amines (JP2000-86863A), imidazoles (JP 2000-178332A), and phosphines (JP 2003-82099A) have been reported. U.S. Pat. No. 6,225,440 B1 discloses Lewis acids, such as PCl₅, TiCl₄, and AlCl₃ as highly active curatives for the polymerization of benzoxazines. However, in practical applications, such strong Lewis acids negatively contribute to the final polymerization result and its practical properties. For example deterioration of chemical resistance and physical properties of the cured material may appear. Additionally, Lewis acids, such as PCl₅, TiCl₄, AlCl₃ are highly sensitive to moisture and could cause the formation of volatile, toxic and/or corrosive impurities.

Alternative curatives based on metal-ligand complexes have also been reported. WO 2008/0348142 A2 discloses several modified acetylacetonate metal complexes as catalysts/curatives for the low temperature curing of benzoxazine-containing compositions.

However taking into account that some of the aforementioned curatives are highly reactive and could cause the partial polymerization of benzoxazine-containing compositions even at temperatures of up to 25° C., it would be desirable to provide alternative curatives that are less reactive at temperatures of up to 25° C. but still reactive enough to cure/polymerize said benzoxazine-containing compositions at temperatures of 180° C. or less.

These alternative curatives would allow providing benzoxazine-containing compositions which exhibit a longer pot-life and a longer open-time at temperatures of up to 25° C.

Additionally, some of the aforementioned curatives could negatively contribute to the thermal stability of the benzoxazine-containing compositions or to the thermal stability of the cured reaction products of the benzoxazine-containing compositions. As a result an undesired weight loss may occur, especially during the polymerization/curing reaction of benzoxazine-containing compositions.

Notwithstanding the state of technology, it would be desirable to provide new benzoxazine-based compositions, which can be cured efficiently in an environmentally friendly process at temperatures of up to 180° C. and which exhibit a long pot-life and a long open-time at temperatures of up to 25° C. Moreover, it would be desirable to provide new benzoxazine-based compositions, which exhibit a high thermal stability and a minimal weight loss during curing.

### SUMMARY OF THE INVENTION

The inventors of the present invention surprisingly found, that curable compositions, comprising specific meta-substituted aromatic compounds and benzoxazine compounds can be cured efficiently at temperatures of 180° C. or less in an environmentally friendly process. Moreover these compositions exhibit a long pot-life, a long open-time at temperatures of up to 25° C., a high thermal stability and a minimal weight loss during curing.

Therefore, the invention relates to a curable composition, comprising

a) at least one meta-substituted aromatic compound of formula (I),

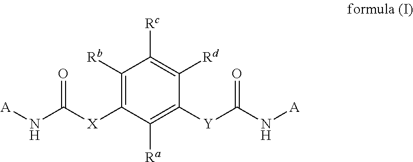

wherein
A is a residue obtained by removing one isocyanate group of an monoisocyanate, or
A is an oligomeric or polymeric residue, comprising at least one repeating unit of formula (II),

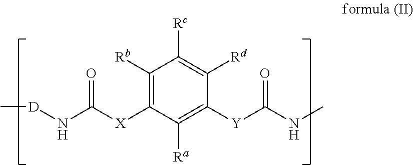

X and Y independently are selected from the group consisting of NR′, O and S, wherein
R′ is hydrogen or a residue selected from the group consisting of aliphatic, heteroaliphatic, araliphatic, heteroaraliphatic, aromatic and heteroaromatic residues,
D is a divalent residue obtained by removing the two isocyanate groups of a diisocyanate,
R^{a}, R^{b}, R^{c} and R^{d} independently are selected form hydrogen, nitro, halogen, carboxyl, carboxylic ester groups, C₁-C₄₀ alkyl groups, C₁-C₄₀ alkoxy groups, C₃-C₄₀ cycloalkyl groups, C₃₋₄₀ alkenyl groups, C₃₋₄₀ alkynyl groups, C₆-C₄₀ aryl groups or C₇-C₄₀ aralkyl groups; and

b) at least one benzoxazine compound.

The curable compositions are in particular suitable as adhesives, sealants, coatings and matrices for the preparation of reinforced material such as prepregs and towpregs and/or can be used in injection molding or extrusion processes.

Therefore it is another object of the present invention to provide an adhesive, sealant or coating, comprising or consisting of the curable composition of the present invention.

Furthermore the invention relates to a cured reaction product of the curable composition of the present invention, in particular a cured reaction product comprising a layer or bundle of fibers. It is further provided a process of preparing such material.

In another object of the present invention the at least one meta-substituted aromatic compound a) of formula (I) is used as a curative for polymerizable compositions, comprising at least one benzoxazine compound, preferably selected from the group consisting of N-alkyl and/or N-alkenyl benzoxazine compounds.

The present invention will be more fully understood by a reading of the following detailed description of the invention.

### DETAILED DESCRIPTION OF THE INVENTION

As noted above, the curable composition of the present invention comprises at least one meta-substituted aromatic compound of formula (I),

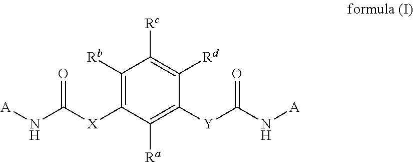

wherein A is a residue obtained by removing one isocyanate group of a monoisocyanate, or
A is an oligomeric or polymeric residue, comprising at least one repeating unit of formula (II),

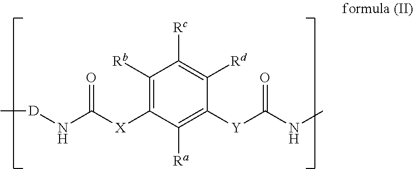

X and Y independently are selected from the group consisting of NR′, O and S, wherein
R′ is hydrogen or a residue selected from the group consisting of aliphatic, heteroaliphatic, araliphatic, heteroaraliphatic, aromatic and heteroaromatic residues,
D is a divalent residue obtained by removing the two isocyanate groups of a diisocyanate, R^{a}, R^{b}, R^{c} and R^{d} independently are selected from hydrogen, nitro, halogen, such as fluorine, chlorine, bromine, or iodine, carboxyl, carboxylic ester groups, C₁-C₄₀ alkyl groups, C₁-C₄₀ alkoxy groups, C₃-C₄₀ cycloalkyl groups, C₃-C₄₀ alkenyl groups, C₃₋₄₀ alkynyl groups, C₆-C₄₀ aryl groups or C₇-C₄₀ aralkyl groups.

The term “C₁₋₄₀ alkyl” as used in the present invention denotes branched and unbranched alkyl groups with 1 to 40 carbon atoms. Preferred are alkyl groups with 1 to 4 carbon atoms. Examples include: methyl, ethyl, n-propyl, iso-propyl, n-butyl, iso-butyl, sec-butyl, tert-butyl, n-pentyl, iso-pentyl, neo-pentyl or hexyl. The definitions propyl, butyl, pentyl and hexyl include all possible isomeric forms of the groups in question. Thus, for example, propyl includes n-propyl and iso-propyl, butyl includes iso-butyl, sec-butyl and tert-butyl etc. Unless otherwise stated, the alkyl groups may be substituted by one or more groups, preferably selected from methyl, ethyl, iso-propyl, tert-butyl, hydroxy, fluorine, chlorine, bromine and iodine.

The term “C₁₋₄₀ alkoxy” as used in the present invention denotes branched and unbranched alkoxyl groups with 1 to 40 carbon atoms. Preferred are alkoxy groups with 1 to 4 carbon atoms. Examples include: methoxy, ethoxy, n-propoxy, iso-propoxy, n-butoxy, iso-butoxy, sec-butoxy, tert-butoxy, n-pentoxy, iso-pentoxy, neo-pentoxy.

The term “C₃₋₄₀ cycloalkyl” as used in the present invention denotes cyclic alkyl groups with 3 to 40 carbon atoms. Examples include: cylopropyl, cyclobutyl, cyclopentyl or cyclohexyl. Unless otherwise stated, the cyclic alkyl groups may be substituted by one or more groups preferably selected from among methyl, ethyl, iso-propyl, tert-butyl, hydroxy, fluorine, chlorine, bromine and iodine.

The term “C₃₋₄₀ alkenyl” as used in the present invention denotes branched and unbranched alkenyl groups with 3 to 40 carbon atoms. Preferred are alkenyl groups with 3 to 5 carbon atoms. Examples include: propenyl, butenyl, pentenyl, or hexenyl. Unless otherwise stated, the definitions propenyl, butenyl, pentenyl and hexenyl include all possible isomeric forms of the groups in question. Thus, for example, propenyl includes 1-propenyl and 2-propenyl, butenyl includes 1-, 2- and 3-butenyl, 1-methyl-1-propenyl, 1-methyl-2-propenyl etc.

The term “C₃₋₄₀ alkynyl” as used in the present invention denotes branched and unbranched alkynyl groups with 3 to 40 carbon atoms. Preferred are alkynyl groups with 3 to 5 carbon atoms. Examples include: propynyl, butynyl, pentynyl or hexynyl. Unless otherwise stated, the definitions propynyl, butynyl, pentynyl and hexynyl include all possible isomeric forms of the groups in question. Thus, for example, propynyl includes 1-propynyl and 2-propynyl, butynyl includes 1-, 2- and 3-butynyl, 1-methyl-1-propynyl, 1-methyl-2-propynyl etc.

The term “C₆-C₄₀ aryl” as used in the present invention denotes aromatic ring systems with 6 to 40 carbon atoms. Examples include: phenyl, naphthyl and anthracenyl, the preferred aryl group being phenyl and napthyl. Unless otherwise stated, the aromatic groups may be substituted by one or more groups preferably selected from among methyl, ethyl, iso-propyl, tert-butyl, hydroxy, alkoxy, such as methoxy or ethoxy, fluorine, chlorine, bromine, iodine and nitro.

The term “C₇₋₄₀ aralkyl” as used in the present invention denotes branched and unbranched alkyl groups with 1 to 30 carbon atoms which are substituted by an aromatic ring system with 6 or 10 carbon atoms. Examples include: benzyl, 1- or 2-phenylethyl. Unless otherwise stated, the aromatic groups may be substituted by one or more groups preferably selected from among methyl, ethyl, iso-propyl, tert-butyl, hydroxy, fluorine, chlorine, bromine and iodine.

The meta-substituted aromatic compound a) of the present invention is a curative/catalyst, which initiates and/or accelerates the curing reaction of the curable composition, even at temperatures of 180° C. or less.

In addition, said meta-substituted aromatic compounds allow producing benzoxazine-containing compositions (curable compositions) which exhibit a long pot-life, a long open-time at temperatures of up to 25° C., and a high thermal stability. Moreover the meta-substituted aromatic compounds of formula (I) can decrease the weight loss of the inventive curable compositions during the curing process.

The term “pot-life” as used in the present invention refers to the length of time a curable composition retains a viscosity low enough for it to be suitable for processing.

The term “open-time” as used in the present invention refers to the elapsed time between the mixture of the curable composition to the curing.

In one embodiment of the present invention the residue A is obtained by removing one isocyanate group of a monoisocyanate, preferably selected from monoisocyanates of general formula A-NCO.

Monoisocyanate for the purpose of this invention can be selected from aliphatic, heteroaliphatic, cycloaliphatic, heterocycloaliphatic, araliphatic, aromatic and/or heteroaromatic monoisocyanates.

Exemplary monoisocyanates include without limitation, ethyl monoisocyanate, propyl monoisocyanate, butyl monoisocyanate, pentyl monoisocyanate, hexyl monoisocyanate, heptyl monoisocyanate, isophorone monoisocyanate, phenyl monoisocyanate, 3,5-dimethylphenyl monoisocyanate, naphthyl monoisocyanate and combinations thereof.

In a preferred embodiment of the present invention the residue A is obtained by removing one isocyanate group of an aromatic monoisocyanate. The resulting meta-substituted aromatic compounds a) of formula (I) having a residue A derived from an aromatic monoisocyanate are highly active curatives/catalysts for the curing/polymerization reaction of benzoxazine compounds.

Even N-alkyl and/or N-alkenyl benzoxazine compounds can be cured efficiently in an environmentally friendly process at temperatures of 180° C. or less, such as temperatures from 90° C. to 160° C., by using the aforementioned meta-substituted aromatic compounds a) of formula (I), preferably having a residue A derived from an aromatic monoisocyanate as a catalyst/curative.

Moreover, said catalysts/curatives can improve the thermal stability of the inventive curable composition, even if N-alkyl and/or N-alkenyl benzoxazine compounds are present in the curable composition.

In a particular preferred embodiment of the present invention residue A in formula (I) is selected from monovalent aromatic residues of

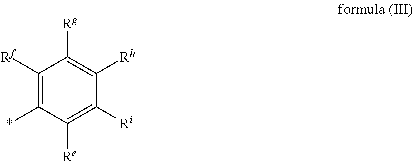

wherein R^{e}, R^{f}, R^{g}, R^{h} and Rⁱ independently are selected from hydrogen, nitro, halogen, such as fluorine, chlorine, bromine, or iodine, carboxyl, carboxylic ester groups, C₁-C₄₀ alkyl groups, C₁-C₄₀ alkoxy groups, C₃-C₄₀ cycloalkyl groups, C₃₋₄₀ alkenyl groups, C₃₋₄₀ alkynyl groups, C₆-C₄₀ aryl groups or C₇-C₄₀ aralkyl groups.

The electronic nature and catalytic activity of the meta-substituted aromatic compound of formula (I) can easily be controlled by varying the substituents R^{e}, R^{f}, R^{g}, R^{h} and Rⁱ on the aromatic ring system.

Preferably at least four of the five substituents R^{e}, R^{f}, R^{g}, R^{h} and Rⁱ are hydrogen and more preferably R^{e}, R^{f}, R^{g}, R^{h} and Rⁱ are all hydrogen.

Preferred monovalent aromatic residues A are selected from the following structures:

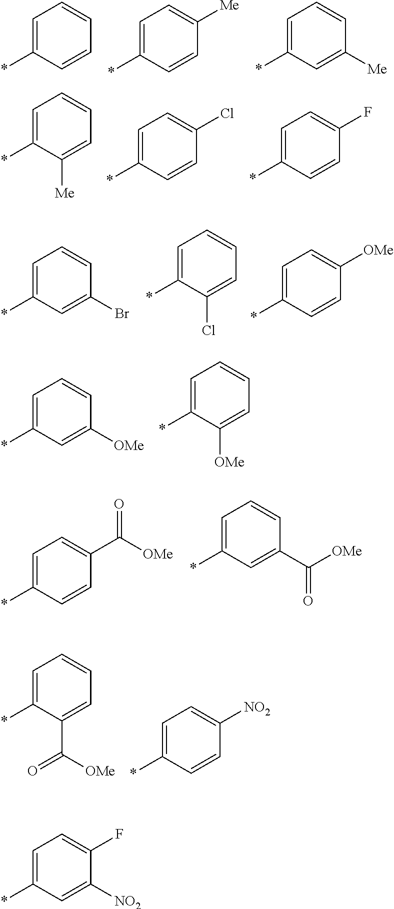

In an alternative embodiment of the present invention the residue A is an oligomeric or polymeric residue, comprising at least one repeating unit of formula (II),

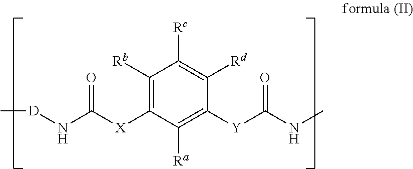

X and Y independently are selected from the group consisting of NR', O and S, wherein
R′ is hydrogen or a residue selected from the group consisting of aliphatic, heteroaliphatic, araliphatic, heteroaraliphatic, aromatic and heteroaromatic residues,
D is a divalent residue obtained by removing the two isocyanate groups of a diisocyanate, R^{a}, R^{b}, R^{c} and R^{d} independently are selected from hydrogen, C₁-C₄₀ alkyl groups, C₃-C₄₀ cycloalkyl groups, C₃₋₄₀ alkenyl groups, C₃₋₄₀ alkynyl groups, C₆-C₄₀ aryl groups or C₇-C₄₀ aralkyl groups.

The term oligomeric residue as used herein refers to a residue A, which comprises from 1 to 10 repeating units of formula (II).

The term polymeric residue as used herein refers to a residue A, which comprises at least 11 repeating units of formula (II).

The oligomeric or polymeric residue A can be linear or branched and the weight average molecular weight of said residue A is preferably in the range of 200 g/mol to 2000000 g/mol, more preferably in the range of 500 g/mol to 1000000 g/mol, particularly preferably in the range of 1000 g/mol to 100000 g/mol and very particularly preferably in the range of 2000 g/mol to 10000 g/mol.

In a preferred embodiment, the repeating unit(s) of formula (II) constitute(s) at least 20 percent by weight, preferably at least 30 percent by weight, more preferably at least 50 percent by weight, particularly preferably at least 70 percent by weight, and very particularly preferably at least 90 percent by weight of the oligomeric or polymeric residue A.

The oligomeric or polymeric residue A can be selected from monovalent oligomeric or polymeric residues of formula (IV),

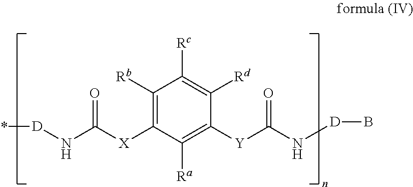

wherein n is an integer of 1 to 10000 and B is an isocyanate group or a monovalent residue of formula (V),

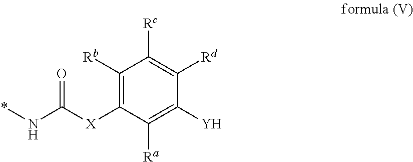

and X, Y, D, R^{a}, R^{b}, R^{c} and R^{d} are defined as above.

The integer n preferably ranges from 2 to 5000, more preferably from 10 to 2500, and particularly preferably 100 to 1000.

The divalent residue D in formula (II) is obtained by removing two isocyanate groups of a diisocyanate, preferably selected from diisocyanates of general formula OCN-D-NCO.

Diisocyanates for the purposes of this invention can be selected from aliphatic, heteroaliphatic, cycloaliphatic, heterocycloaliphatic, araliphatic, aromatic and/or heteroaromatic diisocyanates, preferably having a molecular weight of about 160 g/mol to 500 g/mol.

Useful diisocyanates include, ethylene diisocyanate, trimethylene diisocyanate, tetramethylene diisocyanate, pentamethylene diisocyanate, hexamethylene diisocyanate, heptamethylene diisocyanate, octamethylene diisocyanate, decamethylene diisocyanate, dodecamethylene diisocyanate, tetradecamethylene diisocyanate, hexadecamethylene diisocyanate, octadecamethylene diisocyanate, eicosamethylene diisocyanate, cyclohexamethylene diisocyanate, cyclopenthalene diisocyanate, or cyclohepthalene diisocyanate, or bis-cyclohexylene, cyclohexylmethylene diisocyanate, tetramethylxylylene diisocyanate, phenyl diisocyanate, toluene diisocyanate (such as, 2,4-diisocyanatotoluene and 2,6-diisocyanatotoluene), 4,4′-methylene diphenyl diisocyanate, 4,4′-diphenylene methane diisocyanate, dianisidine diisocyanate, 1,5-naphthalene diisocyanate, 1,8-naphthalene diisocyanate (1,8-NDI), 4,4′-diphenyl ether diisocyanate, p-phenylene diisocyanate, 4,4′-dicyclo-hexylmethane diisocyanate, 1,3-bis-(isocyanatomethyl)cyclohexane, cyclohexylene diisocyanate, tetrachlorophenylene diisocyanate, 2,6-diethyl-p-phenylenediisocyanate, 3,5-diethyl-4,4′-diisocyanatodiphenyl-methane, tetramethylene diisocyanate, hexamethylene diisocyanate, ethylene diisocyanate, cyclohexylene diisocyanate, nonamethylene diisocyanate, octadecamethylene diisocyanate, 2-chloropropane diisocyanate, 2,2′-diethylether diisocyanate, 3-(dimethylamine) pentane diisocyanate, tetrachlorophenylene diisocyanate-1,4,3-heptane diisocyanate, transvinylene diisocyanate, 1,6-diisocyanatohexane, 3,5,5-trimethyl-1-isocyano-3-isocyanatomethylcyclohexane (isophorene diisocyanate), N,N′,N′″-tri-(6-isocyanatohexyl)-biuret, 2,2,4-trimethyl-1,6-diisocyanatohexane, m-tetramethylxylene diisocyanate 1-methyl-2,4-diisocyanatocyclohexane, 4,4′-diisocyanatodicyclohexylmethane, trimeric isophorene diisocyanate, trimeric hexane diisocyanate and methyl 2,6-diisocyanatohexanoate and combinations thereof.

In a preferred embodiment of the present invention residue D in formula (II) is obtained by removing two isocyanate groups from 2,4-toluene diisocyanate, 2,4′-methylenediphenyl diisocyanate, 4,4′-methylene diphenyl diisocyanate, hexamethylene diisocyanate, m-tetramethylxylene diisocyanate, or isophorone diisocyanate.

In another embodiment of the present invention the at least one meta-substituted aromatic compound a) of formula (I) comprises at least one urethane group. More preferably all X and Y in formulae (I), (II), (IV) and (V) are O.

The electronic nature and catalytic activity of the meta-substituted aromatic compound of formula (I), (II), (IV) and or (V) can easily be controlled by varying the substituents R^{a}, R^{b}, R^{c}, and R^{d} on the aromatic ring system. Preferably at least three of the four substituents R^{a}, R^{b}, R^{c}, and R^{d} are hydrogen and more preferably R^{a}, R^{b}, R^{c}, and R^{d} are all hydrogen.

Specific examples of suitable meta-substituted aromatic compounds a) of formula (I) include:

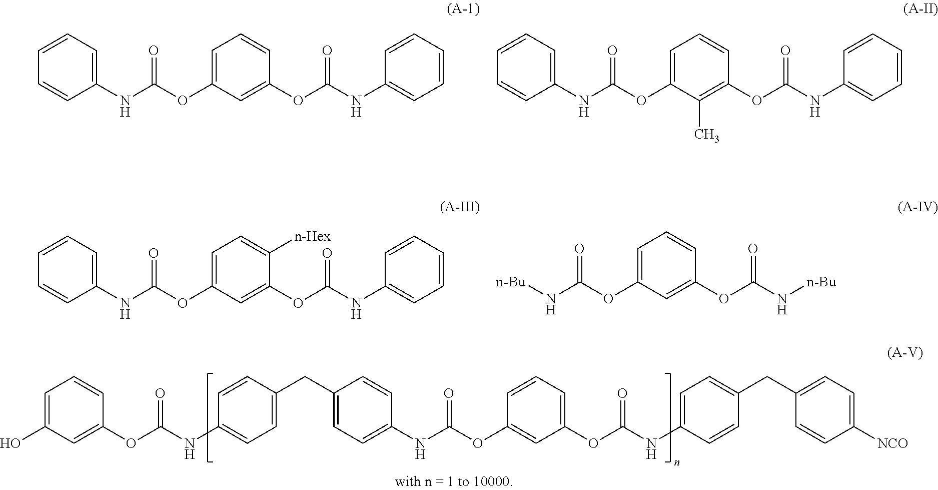

The at least one meta-substituted aromatic compound a) of formula (I) or mixtures of different meta-substituted aromatic compounds a) of formula (I) can be included in an amount in the range of 0.1 to 20 percent by weight, such as 0.2 to 10 percent by weight, desirably in an amount of 0.3 to 5 percent by weight, and more desirably in an amount of 0.5 to 1.5 percent by weight, based on the total amount of the curable composition of the present invention.

The at least one meta-substituted aromatic compound a) of the present invention can be prepared according to any method. One preferred method comprises the step of reacting at least one aromatic compound of

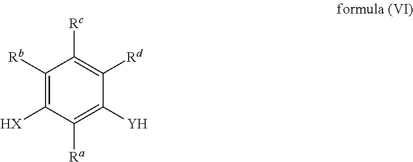

with at least one monoisocyanate of the following formula:

A-NCO

and/or with at least one diisocyanate of the following formula:

OCN-D-NCO

wherein X, Y, R^{a}, R^{b}, R^{c}, R^{d}, A and D are defined as above.

Examples of aromatic compounds of formula (VI) include compounds of formula (VIa)

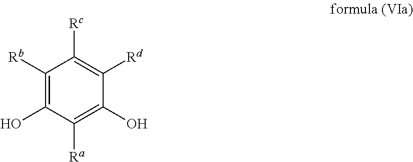

wherein R^{a}, R^{b}, R^{c}, and R^{d} are defined as above. More preferably at least three of the four substituents R^{a}, R^{b}, R^{c}, and R^{d} in formula (VIa) are hydrogen.

In a particular preferred method of preparing the meta-substituted aromatic compound of the present invention resorcinol is reacted with at least one monoisocyanate of the following formula:

A-NCO

and/or with at least one diisocyanate of the following formula:

OCN-D-NCO

wherein A and D are defined as above.

A similar method of preparing meta-substituted aromatic compounds a) of the present invention is described in U.S. patent application No. 2007/0205393 A1, where said compounds are used in rubber compound formulations and fabric dipping formulations for treating fibers, filaments, fabrics or cords.

A further component of the curable composition of the present invention is a benzoxazine compound.

The benzoxazine compound can be any curable monomer, oligomer or polymer comprising at least one benzoxazine moiety. Preferably monomers containing up to four benzoxazine moieties are employed as the benzoxazine compound in form of single compounds or mixtures of two or more different benzoxazines.

In the following a broad spectrum of different suitable benzoxazine compounds, containing one to four benzoxazine moieties are presented.

One possible benzoxazine compound may be embraced by the following structure (B-I):

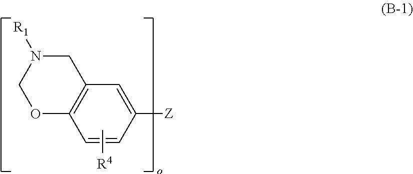

wherein o is 1 to 4, Z is selected from the group consisting of the group consisting of a direct bond (when o is 2), alkyl (when o is 1), alkylene (when o is 2-4), carbonyl (when o is 2), oxygen (when o is 2), thiol (when o is 1), sulfur (when o is 2), sulfoxide (when o is 2), and sulfone (when o is 2), each R¹ is independently selected from hydrogen, alkyl, alkenyl or aryl, and each R⁴ is independently selected from hydrogen, halogen, alkyl and alkenyl or R⁴ is a divalent residue creating a naphthoxazine residue out of the benzoxazine structure.

More specifically, within structure (B-I) the benzoxazine compound may be embraced by the following structure (B-II):

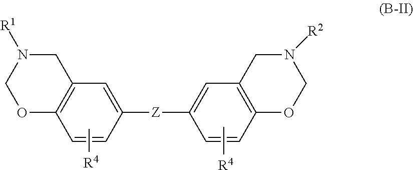

where Z is selected from a direct bond, CH₂, C(CH₃)₂, C═O, O, S, S═O and O═S═O, R¹ and R² are the same or different and are selected from hydrogen, alkyl, such as methyl, ethyl, propyls and butyls, alkenyl, such as allyl, and aryl, and R⁴ are the same or different and defined as above.

Representative benzoxazine compounds within structure (B-II) include:

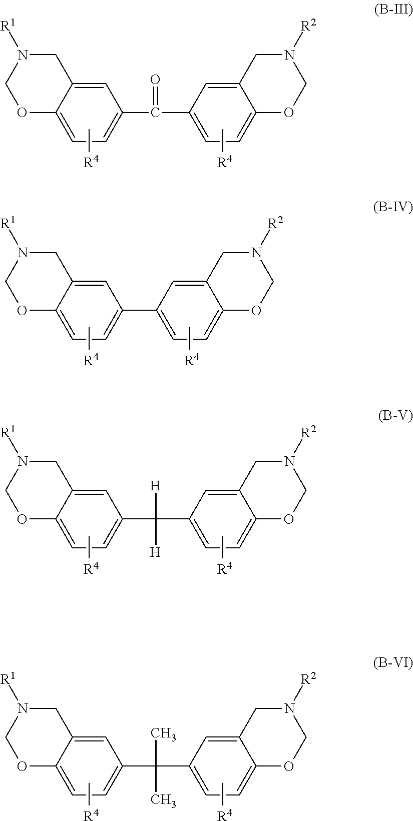

wherein R¹, R² and R⁴ are as defined above.

Alternatively, the benzoxazine compound may be embraced by the following structure (B-VII):

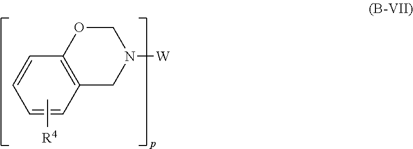

wherein p is 2, W is selected from biphenyl, diphenyl methane, diphenyl isopropane, diphenyl sulfide, diphenyl sulfoxide, diphenyl sulfone, and diphenyl ketone, and R⁴ is selected from hydrogen, halogen, alkyl and alkenyl.

Though not embraced by structures (B-I) or (B-VII) additional benzoxazine compounds are within the following structures:

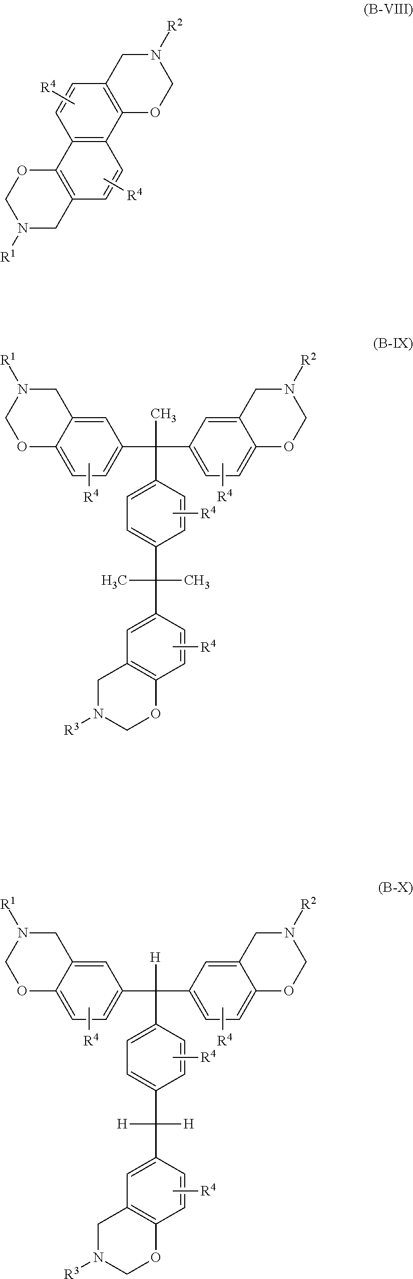

wherein R¹, R² and R⁴ are as defined above, and R³ is defined as R¹, R² or R⁴.

Specific examples of the above generically described benzoxazines

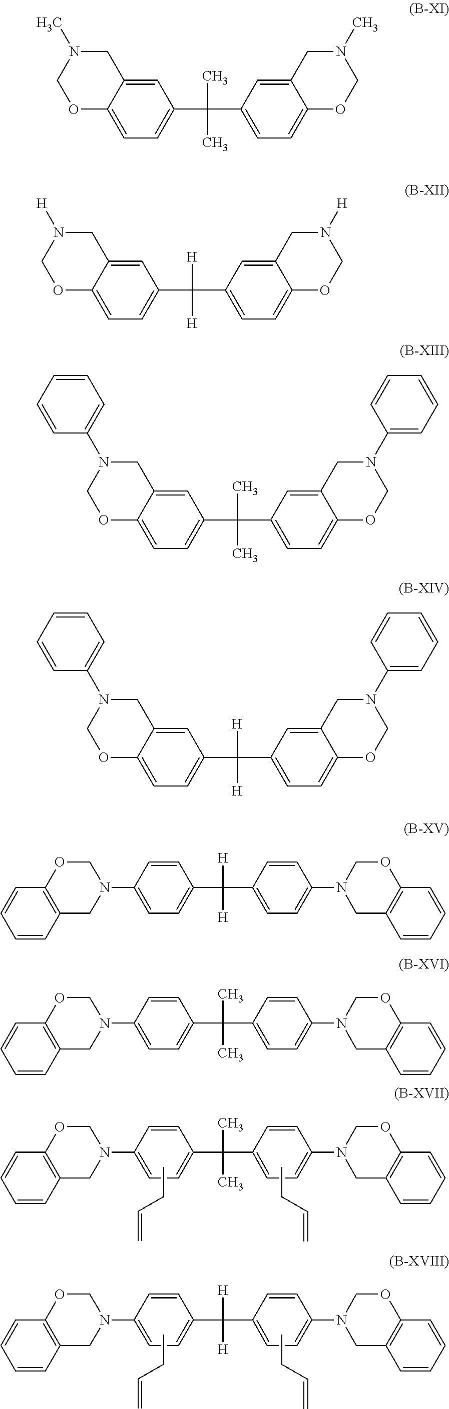

In the present invention combinations of multifunctional benzoxazines and monofunctional benzoxazines, or combinations of one or more multifunctional benzoxazines or one or more monofunctional benzoxazines can be used.

Examples of monofunctional benzoxazine compounds may be embraced by the following structure (B-XIX):

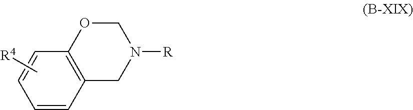

wherein R is alkyl, such as methyl, ethyl, propyl and butyl, alkenyl or aryl with or without substitution on one, some or all of the available substitutable sites, and R⁴ is selected from hydrogen, halogen, alkyl and alkenyl, or R⁴ is a divalent residue creating a naphthoxazine residue out of the benzoxazine structure.

For instance, monofunctional benzoxazine compounds may be embraced by general structure (B-XX):

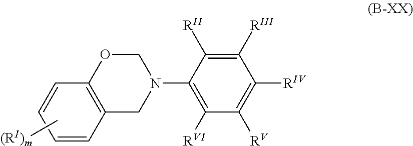

where in this case R^{I} is selected from alkyl, alkenyl, each of which being optionally substituted or interrupted by one or more O, N, S, C═O, COO, and NHC═O, and aryl; m is 0 to 4; and R^{I}, R^{II}, R^{IV}, R^{V} and R^{VI} are independently selected from hydrogen, alkyl, alkenyl, each of which being optionally substituted or interrupted by one or more O, N, S, C═O, COOH, and NHC═O, and aryl.

Specific examples of such a monofunctional benzoxazine compounds are:

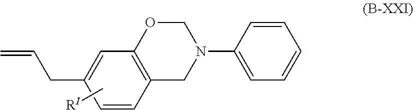

where R^{I} is as defined above; or

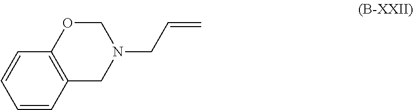

In a preferred embodiment of the present invention the at least one benzoxazine compound b) is selected from the group consisting of N-alkyl and/or N-alkenyl benzoxazine compounds.

The term “N-alkyl benzoxazine compound” as used herein refers to any benzoxazine compound carrying an alkyl residue directly bound at the benzoxazine nitrogen atom.

The term “N-alkenyl benzoxazine compound” as used herein refers to any benzoxazine compound carrying an alkenyl residue directly bound at the benzoxazine nitrogen atom.

One group of N-alkyl or N-alkenyl benzoxazine compounds of the present invention may be embraced by the following structure:

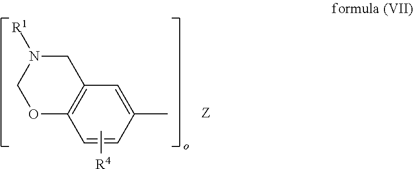

wherein o is 1 to 4, Z is selected from the group consisting of a direct bond (when o is 2), alkyl (when o is 1), alkylene (when o is 2 to 4), carbonyl (when o is 2), oxygen (when is 2), thiol (when o is 1), sulfur (when o is 2), sulfoxide (when o is 2), and sulfone (when o is 2), each R¹ is independently selected from alkyl groups or alkenyl groups, and each R⁴ is independently selected from hydrogen, halogen, alkyl and alkenyl or R⁴ is a divalent residue creating a naphthoxazine residue out of the benzoxazine structure.

Preferred N-alkyl benzoxazine compounds and/or preferred N-alkenyl benzoxazine compounds are embraced by benzoxazine compounds of formula (B-II) to (B-VI) and (B-VIII) to (B-X), wherein the residues R¹, R² and, if present, R³ are selected from alkyl groups or alkenyl groups, such as methyl, ethyl, propyl, vinyl or allyl.

In preferred embodiments of the present invention, the curable composition comprises different benzoxazine compounds, such as mixtures of different N-alkyl benzoxazine compounds and/or mixtures of at least one N-alkyl benzoxazine compound and at least one N-alkenyl benzoxazine compound.

It is preferred to use N-alkyl and/or N-alkenyl benzoxazine compounds in the curable composition of the present invention, because the at least one meta-substituted aromatic compound a) of formula (I) is capable of catalyzing the curing reaction of said compounds in a very efficient way. Moreover, said catalyst/curative can improve the thermal stability of an inventive curable composition, comprising N-alkyl and/or N-alkenyl benzoxazine compounds.

Benzoxazines are presently available commercially from several sources, including Huntsman Advanced Materials; Georgia-Pacific Resins, Inc.; and Shikoku Chemicals Corporation, Chiba, Japan, the last of which offers among others Bisphenol A-aniline, Bisphenol A-methylamine, Bisphenol F-aniline benzoxazine resins.

If desired, however, instead of using commercially available sources, the benzoxazine compound may typically be prepared by reacting a phenolic compound, preferably selected from monophenols and/or diphenols such as biphenyl-4,4′-diol (also known as “4,4′-Biphenol”), Bisphenol A, Bisphenol P, Bisphenol M, Bisphenol F, Bisphenol S, Bisphenol AP, Bisphenol E, 4,4′-oxydiphenol, 4,4′-thiodiphenol, bis(4-hydroxyphenyl)methanone, biphenyl-2,2′-diol, 4,4′-(cyclohexane-1,1-diyl)diphenol or 4,4′-(3,3,5-trimethylcyclohexane-1,1-diyl)diphenol (Bisphenol TMC), with an aldehyde and an alkyl, alkenyl and/or aryl amine. U.S. Pat. No. 5,543,516, hereby expressly incorporated herein by reference, describes a method of forming benzoxazines, where the reaction time can vary from a few minutes to a few hours, depending on reactant concentration, reactivity and temperature. See generally U.S. Pat. Nos. 4,607,091 (Schreiber), 5,021,484 (Schreiber), 5,200,452 (Schreiber) and 5,443,911 (Schreiber).

Any of the before-mentioned benzoxazines may contain partially ring-opened benzoxazine structures.

However, for the purpose of this invention those structures are still considered to be benzoxazine moieties, in particular ring-opened benzoxazine moieties.

The benzoxazine compound is preferably the only curable ingredient in the curable compositions of the present invention. However other curable ingredients or resins can be included, if desired.

The at least one benzoxazine compound b) or mixtures of different benzoxazine compounds b) can be included in an amount in the range of 20 to 99.9 percent by weight, such as 40 to 98 percent by weight, desirably in an amount of 50 to 95 percent by weight, and more desirably in an amount of 60 to 90 percent by weight, based on the total amount of the curable composition of the present invention.

In a particular preferred embodiment the curable composition comprises at least one benzoxazine compound b) or mixtures of different benzoxazine compounds b) in an amount of 60 to 80 percent by weight, such as 60 to 70 percent by weight, based on the total amount of the curable composition of the present invention.

The curable composition of the present invention may comprise at least one benzoxazine compound b) and the at least one meta-substituted aromatic compound a) of formula (I) in an amount, that the molar ratio of benzoxazine moieties to meta-substituted aromatic compounds a) of formula (I) is in the range of 50:50 to 99.9:0.1, preferably in the range of 70:30 to 99.5:0.5.

A higher amount of the meta-substituted aromatic compounds a) of formula (I) could lead to a self-reaction between said meta-substituted aromatic compounds a) and/or between substances which are released from said meta-substituted aromatic compounds a), whereas a smaller amount of the meta-substituted aromatic compounds a) often does not lead to an acceleration of the curing reaction of the inventive curable composition.

The curable compositions of the present invention may further comprise other resin compounds apart from the benzoxazine component, such as epoxy resin components.

The term “epoxy resin”, as used in the present invention, refers to any organic compound having at least two functional groups of oxirane type which can be polymerized by ring opening. The term “epoxy resin” preferably denotes any conventional epoxy resin which is liquid at room temperature (23° C.) or at a higher temperature. These epoxy resins can be monomeric or polymeric, on the one hand, aliphatic, cycloaliphatic, heterocyclic or aromatic, on the other hand.

The epoxy resins used in the present invention may include multifunctional epoxy-containing compounds, such as C₁-C₂₈ alkyl-, poly-phenol glycidyl ethers; polyglycidyl ethers of pyrocatechol, resorcinol, hydroquinone, 4,4′-dihydroxydiphenyl methane (or bisphenol F, such as RE-303-S or RE-404-S available commercially from Nippon Kayaku, Japan), 4,4′-dihydroxy-3,3′-dimethyldiphenyl methane, 4,4′-dihydroxydiphenyl dimethyl methane (or bisphenol A), 4,4′-dihydroxydiphenyl methyl methane, 4,4′-dihydroxydiphenyl cyclohexane, 4,4′-dihydroxy-3,3′-dimethyldiphenyl propane, 4,4′-dihydroxydiphenyl sulfone, and tris(4-hydroxyphenyl)methane; polyglycidyl ethers of transition metal complexes; chlorination and bromination products of the above-mentioned diphenols; polyglycidyl ethers of novolacs; polyglycidyl ethers of diphenols obtained by esterifying ethers of diphenols obtained by esterifying salts of an aromatic hydrocarboxylic acid with a dihaloalkane or dihalogen dialkyl ether; polyglycidyl ethers of polyphenols obtained by condensing phenols and long-chain halogen paraffins containing at least two halogen atoms; phenol novolac epoxy; cresol novolac epoxy; and combinations thereof.

Among the commercially available epoxy resins suitable for use in the present invention are polyglycidyl derivatives of phenolic compounds, such as those available under the tradenames EPON 825, EPON 826, EPON 828, EPON 1001, EPON 1007 and EPON 1009, cycloaliphatic epoxy-containing compounds such as Araldite CY179 from Huntsman or waterborne dispersions under the tradenames EPI-REZ 3510, EPI-REZ 3515, EPI-REZ 3520, EPI-REZ 3522, EPI-REZ 3540 or EPI-REZ 3546 from Hexion; DER 331, DER 332, DER 383, DER 354, and DER 542 from Dow Chemical Co.; GY285 from Huntsman, Inc.; and BREN-S from Nippon Kayaku, Japan. Other suitable epoxy-containing compounds include polyepoxides prepared from polyols and the like and polyglycidyl derivatives of phenol-formaldehyde novolacs, the latter of which are available commercially under the tradenames DEN 431, DEN 438, and DEN 439 from Dow Chemical Company and a waterborne dispersion ARALDITE PZ 323 from Huntsman.

Cresol analogs are also available commercially such as ECN 1273, ECN 1280, ECN 1285, and ECN 1299 or waterborne dispersions ARALDITE ECN 1400 from Huntsman, Inc. SU-8 and EPI-REZ 5003 are bisphenol A-type epoxy novolacs available from Hexion.

Of course, combinations of the different epoxy resins are also desirable for use herein.

If present, the epoxy resin component can be used in the curable composition of the present invention in an amount in the range of 0.1 to 60 percent by weight, more preferably in an amount of 5 to 50, particularly preferably in an amount of 10 to 30 percent by weight, and very particularly preferably in an amount of 15 to 20 percent by weight, based on the total amount of the curable composition.

Additives suitable for use in the present invention include reactive diluents, for example styrene oxide (epoxide of styrene), butyl glycidyl ether, 2,2,4-trimethylpentyl glycidyl ether, phenyl glycidyl ether, cresyl glycidyl ether or glycidyl esters of synthetic, highly branched, mainly tertiary, aliphatic monocarboxylic acids, and oxazoline group containing compounds, tougheners, plasticizers, extenders, microspheres, fillers, such as silica nanoparticles and reinforcing agents, for example coal tar, bitumen, textile fibers, glass fibers, asbestos fibers, boron fibers, carbon fibers, mineral silicates, mica, powdered quartz, hydrated aluminum oxide, bentonite, wollastonite, kaolin, silica, aerogel or metal powders, for example aluminium powder or iron powder, and also pigments and dyes, such as carbon black, oxide colors and titanium dioxide, fire-retarding agents, thixotropic agents, flow control agents, such as silicones, waxes and stearates, which can, in part, also be used as mold release agents, adhesion promoters, antioxidants and light stabilizers, the particle size and distribution of many of which may be controlled to vary the physical properties and performance of the inventive curable composition.

If present, at least one additive or mixtures of different additives can be used in the curable composition of the present invention in an amount in the range of 0.1 to 30 percent by weight, more preferably in an amount of 2 to 20 percent by weight and most preferably in an amount of 5 to 15 percent by weight, based on the total amount of the curable composition.

In one embodiment of the present inventions solvent can be used to lower the viscosity of the curable composition. Preferable solvents are ethers such as diethylether and tetrahydrofuran, ketones such as acetone and ethyl methyl ketone, esters such as ethyl acetate and butyl acetate, chlorinated hydrocarbons such as chloroform and dichloromethane, aromatics such as benzene and chlorobenzene, amides such as dimethylformamide and methylpyroridone, alcohols such as methanol and isopropanol. More preferably, ester-type solvents and ketone-type solvents are used.

In a further embodiment of the present invention the curable composition comprises, based on the total amount of the composition:

a) from 0.1 to 20 percent by weight, more typically from 0.2 to 10 percent by weight, suitably from 0.3 to 5 percent by weight, for example from 0.5 to 1.5 percent by weight of at least one meta-substituted aromatic compound a) of the present invention;

b) from 20 to 99.9 percent by weight, more typically from 40 to 98 percent by weight, suitably from 50 to 95 percent by weight, for example from 60 to 90 percent by weight of at least one benzoxazine compound b);

c) from 0 to 60 percent by weight, more typically from 5 to 50 percent by weight, suitably from 10 to 30 percent by weight, for example from 15 to 25 percent by weight of at least one epoxy resin; and

d) from 0 to 30 percent by weight, more typically from 2 to 20 percent by weight, suitably from 5 to 15 percent by weight, for example from 6 to 12 percent by weight of one or more additives.

In one embodiment of the present invention the curable composition is cured at temperatures from 20° C. to 180° C., preferably from 50° C. to 170° C., more preferably from 120° C. to 160° C. and/or at pressures between 1 to 100 atm, preferably between 1 to 5 atm, and more preferably under atmospheric pressure.

The curable composition of the present invention can also be supplemented with additional curatives without losing their advantages properties in case the use of additional curatives is desired for specific applications.

In this regard Lewis acids, and other known curatives, such as metal halides; organometallic derivatives; metallophorphyrin compounds such as aluminum phthalocyanine chloride; anhydrides, methyl tosylate, methyl triflate, and triflic acid; and oxyhalides can be added to the curable composition of the present invention.

However taking into account that the aforementioned curatives could cause the formation of volatile, toxic and corrosive impurities, curable compositions are preferred that do not comprise the aforementioned additional curatives.

As noted above, the curable compositions of the present invention are in particular suitable as coatings, adhesives, sealants and matrices for the preparation of reinforced material such as prepregs and towpregs and/or can be used in injection molding or extrusion.

In this regard, it is another object of the invention to provide an adhesive, sealant or coating comprising the curable composition of the present invention.

The invention also provides a cured reaction product of the curable composition, in particular cured reaction products containing bundles or layers of fibers infused with the inventive curable composition, and a method of preparing such material.

In this regard, the invention relates to processes for producing a prepreg or a towpregs. One such process includes the steps of (a) providing a layer or bundle of fibers; (b) providing a curable composition of the present invention; (c) joining said curable composition and the layer or bundle of fibers to form a prepreg or a towpregs assembly; and (d) optionally removing excess polymerizable composition from the prepreg or towpreg assembly, and exposing the resulting prepreg or towpreg assembly to elevated temperature and/or pressure conditions sufficient to infuse the layer or bundle of fibers with the curable composition to form a prepreg or a towpregs assembly as the cured reaction product.

Another such process for producing a prepreg or towpreg, includes the steps of (a) providing a layer or bundle of fibers; (b) providing a curable composition of the present invention in liquid form; (c) passing the layer or bundle of fibers through said curable composition to infuse the layer or bundle of fibers with said curable composition; and (d) removing excess of said curable composition from the prepreg or towpreg assembly, and exposing the resulting prepreg or towpreg assembly to elevated temperature and/or pressure conditions sufficient to infuse the layer or bundle of fibers with the curable composition to form a prepreg or a towpregs assembly as the cured reaction product.

Generally, the fiber layer or bundle may be constructed from unidirectional fibers, woven fibers, chopped fibers, non-woven fibers or long, discontinuous fibers.

The fiber chosen may be selected from carbon, glass, aramid, boron, polyalkylene, quartz, polybenzimidazole, polyetheretherketone, polyphenylene sulfide, poly p-phenylene benzobisoaxazole, silicon carbide, phenolformaldehyde, phthalate and napthenoate.

The carbon may be selected from polyacrylonitrile, pitch and acrylic, and the glass is selected from S glass, S2 glass, E glass, R glass, A glass, AR glass, C glass, D glass, ECR glass, glass filament, staple glass, T glass and zirconium oxide glass.

The inventive curable composition (and prepregs and towpregs prepared therefrom) is particularly useful in the manufacture and assembly of composite parts for aerospace and industrial end uses, bonding of composite and metal parts, core and core-fill for sandwich structures and composite surfacing.

The inventive curable composition is also useful as a coating, sealant or adhesive for the electronics industry. Suitable substrates on which the curable compositions of the present invention are applied are metals such as steel, aluminum, titanium, magnesium, brass, stainless steel, galvanized steel, like HDG-steel and EG-steel; silicates such as glass and quartz; metal oxides; concrete; wood; electronic chip material, for instance semiconductor chip material; or polymers such as polyimide films and polycarbonate.

The invention also relates to a method to increase the polymerization rate of a polymerizable composition at temperatures up to 180° C., preferably at temperatures up to 160° C. and more preferably at temperatures from 50° C. to 150° C., steps of which comprise:

a) adding at least one meta-substituted aromatic compound a) of the present invention to a polymerizable composition;

b) subjecting the polymerizable composition to conditions appropriate to polymerize the polymerizable composition,

wherein the polymerizable composition comprises at least one benzoxazine compound, preferably selected from the group consisting of N-alkyl and N-alkenyl benzoxazine compounds.

The term “polymerizable composition” refers to a composition, which comprises at least one benzoxazine compound, such as benzoxazine compounds of formula (B-I) to (B-XXII). Preferred benzoxazine compounds are selected from N-alkyl and/or N-alkenyl benzoxazine compounds, such as N-alkyl and/or N-alkenyl benzoxazine compounds of formula (VII).

The term “polymerization rate” as used herein means an average value of the amounts of a change in polymerization conversion per every unit hour (%/hour) obtained in the first 4 hours after starting the polymerization. The polymerization rate can easily be determined by a man skilled in the art using known techniques, such as GC-analysis, NMR- or IR spectroscopy.

In preferred embodiments of the present invention the polymerization rate is determined at temperatures from 20° C. to 180° C., preferably from 50° C. to 170° C., and more preferably from 120° C. to 150° C. and/or at pressures between 1 to 100 atm, preferably between 1 to 5 atm, and more preferably under atmospheric pressure.

In a particular preferred embodiment of the present invention the polymerizable composition comprises at least one benzoxazine compound in an amount from about 5 to about 100 percent by weight, preferably from about 20 to about 99 percent by weight and more preferably from about 40 to about 95 percent by weight, particularly preferably from about 50 to about 90 percent by weight, and very particularly preferably from about 60 to about 80 percent by weight, based on the total amount of the polymerizable composition.

The polymerizable composition of the present invention can further comprise other curable ingredients, such as epoxy resins and/or additives, such as reactive diluents, tougheners, plasticizers, extenders, microspheres, fillers, pigments, dyes, fire-retarding agents, thixotropic agents, flow control agents, adhesion promoters, antioxidants and/or light stabilizers and/or mixtures or combinations thereof.

If present, the epoxy resin component can be used in the polymerizable composition of the present invention in an amount in the range of 1 to 60 percent by weight, more preferably in an amount of 5 to 50, particularly preferably in an amount of 10 to 30 percent by weight and very particularly preferably in an amount of 15 to 20 percent by weight, based on the total amount of the polymerizable composition.

If present, at least one additive or mixtures of different additives can be used in the polymerizable composition of the present invention in an amount in the range of 0.1 to 30 percent by weight, more preferably in an amount of 2 to 20 percent by weight and most preferably in an amount of 5 to 15 percent by weight, based on the total amount of the polymerizable composition.

Preferably, the polymerizable composition of the present invention is cured at temperatures from 40° C. to 180° C., preferably from 50° C. to 150° C., and more preferably from 120° C. to 140° C. and/or at pressures between 1 to 100 atm, preferably between 1 to 5 atm, and more preferably under atmospheric pressure.

A last object of the present invention is the use of at least one meta-substituted aromatic compound of the present invention as a curative/catalyst for polymerizable compositions, comprising at least one benzoxazine compound, preferably selected from the group consisting of N-alkyl and/or N-alkenyl benzoxazine compounds.

The invention is further illustrated by the following examples.

### EXAMPLES

The following benzoxazine compounds were used in the examples:

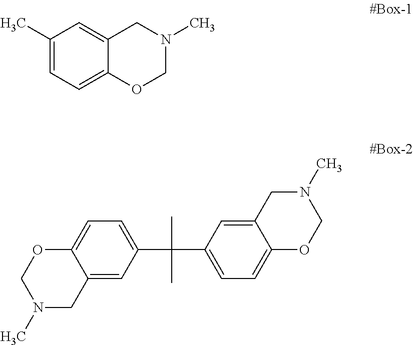

### Example 1.1

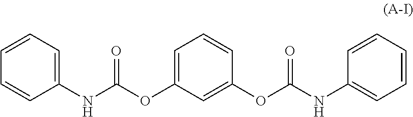

#Box-1 (2.50 g, 15.3 mmol) and the meta-substituted aromatic compound A-I (53.9 mg, 0.155 mmol) were mixed in diethyl ether at 22° C. to obtain a homogeneous formulation. The diethyl ether was removed under reduced pressure at 60° C. for 6 hours. The resulting mixture was divided into five portions and each portion was placed in a test tube.
After degassing, Argon inlets were attached to the test tubes and each test tube was heated in an oil bath at 150° C. for a defined period of time.
From time to time (0.5, 1, 2 hours) these test tubes were taken away from the oil bath one-by-one, and each of the mixture was analyzed by ¹H-NMR to determine the conversion of the benzoxazine compound. The resulting time-conversion relationships are shown in Table 1.

### Example 1.2

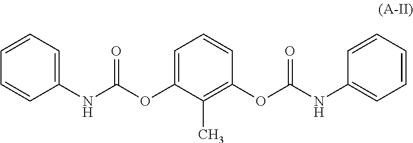

Following the procedure of Example 1.1, except of using the meta-substituted aromatic compound A-II (56.1 mg, 0.155 mmol) instead of A-I, the conversion of #Box-1 was determined. The resulting time-conversion relationships are shown in Table 1.

### Example 1.3

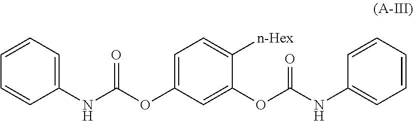

Following the procedure of Example 1.1, except of using the meta-substituted aromatic compound A-III (67.0 mg, 0.155 mmol) instead of A-I, the conversion of #Box-1 was determined. The resulting time-conversion relationships are shown in Table 1.

### Example 1.4

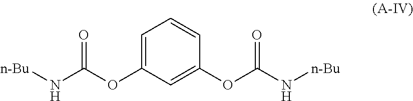

Following the procedure of Example 1.1, except of using the meta-substituted aromatic compound A-IV (47.8 mg, 0.155 mmol) instead of A-I, the conversion of #Box-1 was determined. The resulting time-conversion relationships are shown in Table 1.

### Comparative Example 1.5

Without using any catalyst/curative the conversion of #Box-1 was determined by following the procedure of Example 1.1.

### Comparative Example 1.6

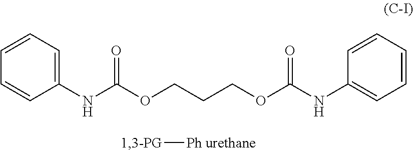

Following the procedure of Example 1.1, except of using a 1,3-propylene glycol urethane of formula (C—I) (48.7 mg, 0.155 mmol) instead of A-I, the conversion of #Box-1 was determined. The resulting time-conversion relationships are shown in Table 1.

TABLE 1
Conversions [%] of #Box-1 at 150° C.

| | | time (h) | | | |
|---|---|---|---|---|---|
| Example | Catalyst/Curative | 0 | 0.5 | 1.0 | 2.0 |
| Example 1-1 | A-I | 0 | 66 | 83 | 89 |
| Example 1-2 | A-II | 0 | 66 | 82 | 88 |
| Example 1-3 | A-III | 0 | 62 | 80 | 87 |
| Example 1-4 | A-IV | 0 | 22 | 47 | 82 |
| Comparative example 1-5 | without | 0 | 9 | 25 | 61 |
| | catalyst/curative | | | | |
| Comparative example 1-6 | 1,3-PG-Ph urethane | 0 | 6 | 23 | 74 |

The results clearly indicate that the polymerization rate of benzoxazine compounds, like N-alkyl benzoxazine compounds, can significantly be increased by using at least one meta-substituted aromatic compound of the present invention as a catalyst/curative. The catalytic activity of comparable aliphatic urethane compounds, like the 1,3-propylene glycol urethane of formula (C—I), is significantly lower than the catalytic activity of the aforementioned meta-substituted aromatic compounds.

### Example 2.1

The meta-substituted aromatic compound A-I (54.3 mg, 0.156 mmol; 5 mol % of #Box-2) and the benzoxazine compound #Box-2 (1.00 g, 2.95 mmol) were mixed in acetone at 22° C. to obtain a homogeneous formulation. The acetone was removed under reduced pressure at 60° C. for 6 hours.

Two sample of the resulting mixture (15.0 mg) were further analyzed by using thermo-gravimetric analyzer (Seiko Instruments Inc. EXTAR 6200 TG).

One sample was heated in the thermo-gravimetric analyzer at 180° C. for 3 hours under a nitrogen atmosphere. The other sample was heated in the thermo-gravimetric analyzer at 200° C. for 3 hours under a nitrogen atmosphere.

For both samples the weight loss during the curing reaction was determined. The results are shown in Table 2.

### Example 2.2

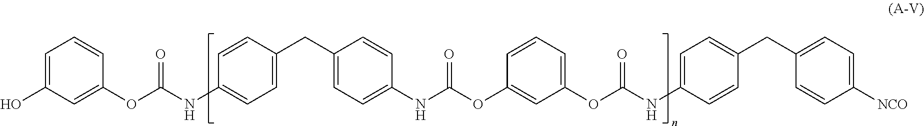

Mixture of different compounds (n ranges from 1 to 10)

Following the procedure of Example 2.1, except of using the polymeric meta-substituted aromatic compound of formula A-V instead of A-I, the weight loss during the curing reaction (3 h, 180° C. and 200° C.) was determined. The results are shown in Table 2.

### Comparative Example 2.3

Without using any catalyst/curative the weight loss during the curing reaction of #Box-2 was determined by following the procedure of Example 2.1. The results are shown in Table 2.

TABLE 2
Weight loss during the curing reaction of #Box-2

| | | Weight loss/% | Weight loss/% |
|---|---|---|---|
| | | during the curing | during the curing |
| | Catalyst/ | reaction at 180° C. | reaction at 200° C. |
| Example | curative | for 3 h | for 3 h |
| Example 2.1 | A-I | 0.9 | 0.9 |
| Example 2.2 | A-V | 0.5 | 0.5 |
| Comparative | none | 1.1 | 1.2 |
| example 2.3 | | | |

The results clearly indicate that the meta-substituted aromatic compounds of the present invention minimize the weight loss during the curing reaction of benzoxazine-based curable compositions.

### Example 3

### Storage Stability

### Example 3.1 and 3.2

#Box-1 (1.63 g, 10.0 mmol) and the meta-substituted aromatic compound A-I (34.8 mg, 0.10 mmol; or 174 mg, 0.50 mmol) were mixed at 22° C. to obtain a homogeneous formulation. The resulting formulation was divided into three portions and each portion was placed in a test tube.

After degassing, Argon inlets were attached to the test tubes and each test tube was stored at 22° C. for a defined period of time. From time to time (0, 72, 144 hours) each of the formulations was analyzed by ¹H-NMR to determine the conversion of the benzoxazine compound #Box-1. The resulting time-conversion relationships are shown in Table 3.

### Comparative Example 3.3 and 3.4

Following the procedure of Example 3.1, except of using resorcinol (11.0 mg, 0.100 mmol; or 55.1 mg, 0.500 mmol) instead of A-I, the conversion of #Box-1 was determined. The resulting time-conversion relationships are shown in Table 3.

### Comparative Example 3.5

Following the procedure of Example 3.1, except of using no catalyst/curative at all, the conversion of #Box-1 was determined. The resulting time-conversion relationships are shown in Table 3.

TABLE 3
Conversions [%] of #Box-1 at 22° C.

| | | time (h) | | |
|---|---|---|---|---|
| Example | Catalyst/Curative | 0 | 72 | 144 |
| Example 3.1 | 1 mol.-% A-I | 0 | 2 | 3 |
| Example 3.2 | 5 mol.-% A-II | 0 | 1 | 2 |
| Comparative | 1 mol.-% resorcinol | 0 | 3 | 5 |
| example 3.3 | | | | |
| Comparative | 5 mol.-% resorcinol | 0 | 17 | 19 |
| example 3.4 | | | | |
| Comparative | without | 0 | <1 | <1 |
| example 3.5 | catalyst/curative | | | |

Resorcinol caused the partial polymerization of the benzoxazine-containing composition at 22° C., whereas the meta-substituted aromatic compounds of the present invention were less reactive at 22° C. and do not initiate a significant curing reaction.

What is claimed is:

1. A curable composition comprising:
a) at least one meta-substituted aromatic compound of formula (I), 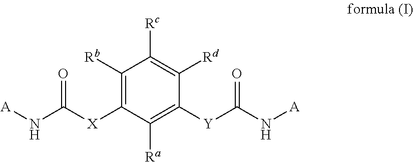
wherein
A is a residue obtained by removing one isocyanate group of an monoisocyanate, or
A is an oligomeric or polymeric residue, comprising at least one repeating unit of formula (II), 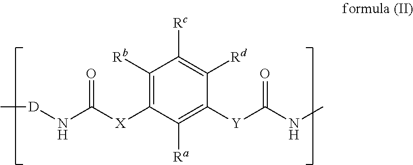
X and Y independently are selected from the group consisting of NR′, O and S, wherein R′ is hydrogen or a residue selected from the group consisting of aliphatic, heteroaliphatic, araliphatic, heteroaraliphatic, aromatic and heteroaromatic residues,
D is a divalent residue obtained by removing the two isocyanate groups of a diisocyanate,
R^{a}, R^{b}, R^{c} and R^{d} independently are selected from hydrogen, nitro, halogen, carboxyl, carboxylic ester groups, C₁-C₄₀ alkyl groups, C₁-C₄₀ alkoxy groups, C₃-C₄₀ cycloalkyl groups, C₃₋₄₀ alkenyl groups, C₃₋₄₀ alkynyl groups, C₆-C₄₀ aryl groups or C₇-C₄₀ aralkyl groups; and
b) at least one benzoxazine compound.

2. The curable composition of claim 1, wherein residue A is obtained by removing one isocyanate group of an aromatic monoisocyanate.

3. The curable composition of claim 1, wherein residue A is selected from monovalent residues of formula (III), 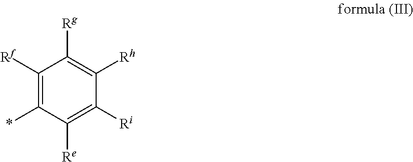
wherein R^{e}, R^{f}, R^{g}, R^{h} and Rⁱ independently are selected from hydrogen, nitro, halogen, carboxyl, carboxylic ester groups, C₁-C₄₀ alkyl groups, C₁-C₄₀ alkoxy groups, C₃-C₄₀ cycloalkyl groups, C₃₋₄₀ alkenyl groups, C₃₋₄₀ alkynyl groups, C₆-C₄₀ aryl groups or C₇-C₄₀ aralkyl groups.

4. The curable composition of claim 3, wherein R^{e}, R^{f}, R^{g}, R^{h} and Rⁱ are hydrogen.

5. The curable composition of claim 1, wherein residue A is selected from monovalent oligomeric or polymeric residues of formula (IV), 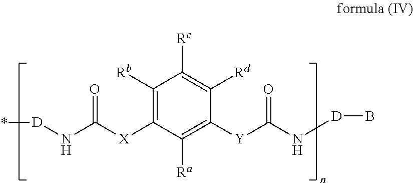
wherein n is an integer of 1 to 10000 and B is an isocyanate group or a monovalent residue of formula (V), 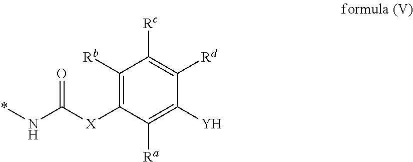
and X, Y, D, R^{a}, R^{b}, R^{c} and R^{d} are defined as in claim 1.

6. The curable composition of claim 1, wherein X and Y in formulae (I), (II), (IV) and (V) are O.

7. The curable composition of claim 1, wherein the benzoxazine compound is selected from the group consisting of N-alkyl and/or N-alkenyl benzoxazine compounds.

8. The composition according to claim 7, wherein the at least one benzoxazine compound is selected from N-alkyl or N-alkenyl benzoxazine compounds 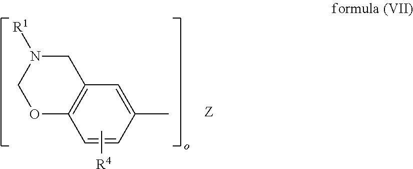
wherein o is 1 to 4, Z is selected from the group consisting of a direct bond (when o is 2), alkyl (when o is 1), alkylene (when o is 2 to 4), carbonyl (when o is 2), oxygen (when is 2), thiol (when o is 1), sulfur (when o is 2), sulfoxide (when o is 2), and sulfone (when o is 2), each R¹ is independently selected from alkyl groups or alkenyl groups, and each R⁴ is independently selected from hydrogen, halogen, alkyl and alkenyl or R⁴ is a divalent residue creating a naphthoxazine residue out of the benzoxazine structure.

9. The curable composition of claim 1, wherein the molar ratio of benzoxazine moieties to meta-substituted aromatic compounds a) as defined in any one of claims 1 to 6 is in the range of 50:50 to 99.9:0.1.

10. The curable composition of claim 1, wherein the at least one meta-substituted aromatic compound is present from 0.1 to 20 percent by weight;
and the at least one benzoxazine compound is present from 20 to 99.9 percent by weight.

11. A cured reaction product of the curable composition of claim 1.

12. A layer or bundle of fibers infused with the curable composition of claim 1 before curing.

13. A process for producing a cured reaction product, steps of which comprise:
a) providing a layer or bundle of fibers;
b) providing the curable composition of claim 1;
c) joining the composition and the layer or bundle of fibers to form an assembly,
d) optionally removing excess curable composition from the assembly;
and exposing the resulting assembly to elevated temperature and/or pressure conditions sufficient to infuse the layer or bundle of fibers with the curable composition to form the cured reaction product.

14. An adhesive, sealant or coating composition comprising the curable composition of claim 10.

15. A method to increase the polymerization rate of a polymerizable composition at temperatures up to 180° C., steps of which comprise:
a) adding at least one meta-substituted aromatic compound a) as defined in claim 1 to a polymerizable composition;
b) subjecting the polymerizable composition to conditions appropriate to polymerize the polymerizable composition,
wherein the polymerizable composition comprises at least one benzoxazine compound, preferably selected from the group consisting of N-alkyl and N-alkenyl benzoxazine compounds.

16. Use of at least one meta-substituted aromatic compound a) as defined in claim 1 as a curative for polymerizable compositions, comprising at least one benzoxazine compound, preferably selected from the group consisting of N-alkyl and/or N-alkenyl benzoxazine compounds.
